(19) United States
(12) Patent Application Publication
(10) Pub. No.: US 2017/0319480 A1
(43) Pub. Date: Nov. 9, 2017

(54) PHARMACEUTICAL FORMULATION

(71) Applicant: Boehringer Ingelheim International GmbH, Ingelheim am Rhein (DE)

(72) Inventors: Detlef MOHR, Biberach an der Riss (DE); Florian SOMMER, Hawangen (DE)

(21) Appl. No.: 15/660,095

(22) Filed: Jul. 26, 2017

Related U.S. Application Data
(63) Continuation of application No. 14/826,240, filed on Aug. 14, 2015.

(30) Foreign Application Priority Data
Aug. 15, 2014 (EP) 14181133.1

(51) Int. Cl. A61K 9/08; A61K 47/46; A61K 47/26; A61K 47/12; A61K 47/02; A61K 31/517; A61K 9/48; A61K 9/00; A61M 5/28

(52) U.S. Cl. CPC A61K 9/08; A61M 5/284; A61K 47/46; A61K 47/26; A61K 47/12; A61K 9/0053; A61K 31/517; A61K 9/4816; A61K 9/0095; A61K 9/009; A61K 47/02

(57) ABSTRACT

The present invention relates to pharmaceutical formulations of highly active drugs with limited shelf-life in aqueous media, suitable to be administered by a caregiver person to a patient avoiding or minimizing the risk of exposure, contact or contamination of the caregiver person with the active product ingredient (API), preferably an EGFR-TKI such as afatinib dimaleate.

### FIELD OF THE INVENTION

The present invention relates to pharmaceutical formulations of highly active drugs with limited shelf-life in aqueous media, suitable to be administered by a caregiver person to a patient avoiding or minimizing the risk of exposure, contact or contamination of the caregiver person with the active product ingredient (API).

### BACKGROUND OF THE INVENTION

Therapy of patients with serious diseases, e.g. of pediatric patients or of disabled patients, often needs support by caregiver persons for administration of medication comprising highly active product ingredient (API), e.g. administration of chemotherapeutic drugs in oncology. If the patient is not able to swallow a solid dosage form of a drug, such as a tablet formulation, there may be the need to administer a liquid formulation of the drug orally. In case there is a stability problem of the API caused by humidity, e.g. the API is susceptible to hydrolytic decomposition, a ready-to-use water based liquid formulation will not be readily available due to insufficient shelf-life for stockpiling but must be prepared on demand. In consequence, a caregiver person must prepare a liquid formulation of the drug starting from a solid formulation upon need, e.g. from a tablet or a powdery formulation, suitable for oral administration to the patient. This may cause serious safety issues to the caregiver since exposure, contact or contamination of the caregiver person with the API of highly active drugs, such as contact with dust generated during processing the solid starting formulation for preparation of the oral solution, should be avoided.

Particularly for pediatric cancer patients there are still unmet needs regarding treatment options, e.g. necessity to develop suitable pharmaceutical formulations of oncological drugs primarily approved for treatment of adult patients. In Europe 15000 children are diagnosed with cancer each year and cancer is the 2nd leading cause of death. Data from the German paediatric cancer registry documented 48379 cancer diagnoses in children between 1980 and 2010 and 17876 between 2001-2010. The median age at diagnosis was 5 years and 11 months. Most frequently diagnosed paediatric tumours comprise leukemias (about 34%), CNS tumours (about 20%) and lymphomas (about 13%).

BIBW 2992 (INN: afatinib) is known as the compound 4-[(3-chloro-4-fluorophenyl)amino]-6-{[4-(N,N-dimethylamino)-1-oxo-2-buten-1-yl]amino}-7-((S)-tetrahydrofuran-3-yloxy)-quinazoline,

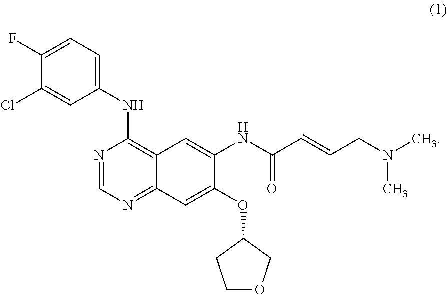 [SMILES: CN(C)C/C=C/C(=O)Nc1cc2c(Nc3ccc(F)c(Cl)c3)ncnc2cc1O[C@H]1CCOC1]

BIBW 2992 is a potent and selective dual inhibitor of erbb1 receptor (EGFR) and erbB2 (Her2/neu) receptor tyrosine kinases. Furthermore, BIBW 2992 was designed to covalently bind to EGFR and HER2 thereby irreversibly inactivating the receptor molecule it has bound to. This compound, salts thereof such as the dimaleate salt (BIBW 2992 MA2), their preparation as well as pharmaceutical formulations comprising BIBW 2992 or a salt thereof, indications to be treated with BIBW 2992 and combinations including BIBW 2992 are disclosed in WO 02/50043, WO 2005/037824, WO 2007/054550 and WO 2007/054551. Solid oral formulations comprising BIBW 2992 are disclosed in WO 2009/147238 and WO 2011/003853. Afatinib is available in a solid oral dosage form as 20 mg, 30 mg, 40 mg and 50 mg film-coated tablets. In WO 2009/147238 is mentioned inter alia that blends comprising a powdery compacted intermediate of BIBW 2992 MA2 may be filled in conventional capsules, e.g. hard gelatin or HPMC capsules.

WO 2008/097658 (Poniard Pharmaceuticals, Inc.) discloses an encapsulated unit dosage form of picoplatin in powdery formulation adapted for oral administration, e.g. filled in hard gelatin, gelatin/PEG or hydroxypropylmethylcellulose (HPMC) capsules.

None of the prior art documents cited discloses the preparation of a ready-to-use liquid formulation by a caregiver, starting from a solid drug formulation, and a safety issue for the caregiver in this connection or in connection with assistance or support for administration of highly active agent to a patient in need of assistance, such as a disabled or a pediatric patient.

BIBW 2992 is suitable for the treatment of tumoral diseases and approved for the treatment of Epidermal Growth Factor Receptor (EGFR) TKI-naive adult patients with locally advanced or metastatic non-small cell lung cancer (NSCLC) with activating EGFR mutations. It is administered as the dimaleate salt (BIBW 2992 MA2). Indications to be treated with BIBW 2992 and combination treatments are disclosed in WO 2007/054550 and WO 2007/054551. For treatment of pediatric patients there is need of a suitable oral liquid formulation comprising BIBW 2992 MA2 as the API which can be easily and safely handled by a caregiver person for administration to the patient. BIBW 2992 MA2 is susceptible against moisture affecting the chemical stability of the API and leading to decrease of the active principle and increase of contamination with hydrolytic decomposition products. Thus a ready to use oral solution of BIBW 2992 MA2 is not feasible as the active substance is not sufficiently stable in solution and prolonged exposure to water should be avoided in preparation of the oral liquid formulation.

One approach to solve this problem could be a solid dosage form of a highly potent compound, specifically BIBW 2992 MA2, for reconstitution to an oral solution or an oral suspension, within an adequate period of time, e.g. within 30 min, that poses no safety risk for a caregiver.

Use of a powder or of dry granules as the solid dosage form is not suitable, since these forms cause safety problems due to generation of dust during preparation of the oral solution, which should be avoided for an oncological product. Uncoated or effervescent tablets also seem to be not suitable since API contact on the surface of dosage form is possible. Furthermore, coated effervescent tablets seem unsuitable from a manufacturing perspective. Use of a coated tablet may be an option but is difficult to process, taking into account that film-coatings provide the tentative risk of inducing stability challenges due to water intake and organic solvent based coatings are typically not preferred, especially for pediatric indications.

Thus the problem underlying the invention is to provide an oral liquid formulation of a highly active drug with a limited shelf-life due to decomposition upon contact with water, e.g. due to hydrolytic decomposition, which can be easily and safely prepared or transformed by a caregiver person into the form ready for administration and safely handled by a caregiver person when administered to a patient in need of treatment, avoiding or minimizing the risk of exposure, contact or contamination of the caregiver person with the API.

In the context of the invention a highly active drug may be understood as a drug with the potential to cause undesirable effects to a person exposed to the drug, either for the therapeutic efficacy of the drug in a healthy person not in need of treatment or for the potential of adverse events or side effects. Basically all drugs have the potential to cause undesirable effects in a person not in need of treatment but there are certain classes of drugs which may cause serious harm to a caregiver person after topic, inhalative or oral contact due to their specific and high potency. Nonexhaustive examples of highly active classes of drugs comprise hormones, corticosteroids, antibiotics, antivirals such as drugs for treatment of HIV or HCV infection, and particularly chemotherapeutics, cytostatic or antiproliferative drugs used in treatment of cancer, such as disclosed in WO 2007/054551. Inhibitors of the erbb1 receptor (EGFR) and/or erbB2 (Her2/neu) receptor tyrosine kinases may be mentioned specifically in this context, such as afatinib, gefitinib, erlotinib, pelitinib, neratinib, HKI-357, CI-1033 (canertinib), WZ 3146, WZ 4002, WZ 8040 (structures of the three WZ compounds disclosed by Wenjun Zhou et al.: Novel mutant-selective EGFR kinase inhibitors against EGFR T790M, in Nature 2009, Vol. 462, 1070-1074), dacomitinib, CO-1686 (CAS Number: 1374640-70-6), AZD9291 (CAS Number: 1421373-65-0), the compounds described in WO 2008/150118 and WO 2011/155793 including HM781-36B, the compounds described in WO 2011/162515.

### SUMMARY OF THE INVENTION

The problem underlying the invention has been addressed by development of a pharmaceutical capsule comprising the highly active drug in a powdery formulation, intended to be dissolved in a suitable solvent as a reconstitution medium for preparation of an oral solution ready for oral administration.

The capsule for oral solution is sufficiently stable and allows preparing an oral solution without exposing caregivers to dust containing the API. Large capsules, e.g. size 00 with an approximate length of 23.3 mm, have been chosen to avoid inadvertent swallowing of the capsules. In order to mask uncomfortable taste, e.g. bitter taste, of the API, sweetener and flavors are added to the solvent. Suitable choice of sweetener and flavors can be confirmed by E-tongue measurements. For example, two capsules can be dissolved in 100 ml solvent, resulting in a 4 mg/ml oral solution. Dosing and administration are planned to be done using an oral syringe suitable for the intended volume.

A first aspect of the invention in its first and broadest embodiment is directed to a pharmaceutical kit developed for patients who cannot swallow tablets, which can be easily and safely handled by a caregiver person supporting administration of the drug to the patient, comprising
(i) at least one water-soluble pharmaceutical capsule containing a powder formulation of a drug comprising an API susceptible to hydrolytic decomposition,
(ii) 50 to 250 ml of a suitable pharmaceutically acceptable solvent as a reconstitution medium contained in a pharmaceutically acceptable container, such as a bottle, 5 to 300% oversized by volume, for preparation of an oral solution comprising the API ready for administration with a shelf-life of the oral solution of up to 6 months at ambient temperature, and, optionally but preferably
(iii) an oral syringe of suitable volume and graduation which can be connected with the bottle via an adapter plug, for dosing and administration,

and, optionally,
(iv) handling instructions comprising preparation of the oral API solution, measurement, withdrawal and administration of a dose.

The expression “shelf-life of the oral solution at ambient temperature” is synonym with an in-use stability of the oral solution at temperatures ranging from about 2° C.-25° C., including refrigerated conditions (2° C.-8° C.) and room temperature (20° C.-25° C.).

Preferably the drug is a highly active drug as mentioned hereinbefore.

A second aspect of the invention in its first and broadest embodiment is directed to
(ii) a suitable pharmaceutically acceptable solvent as a reconstitution medium for preparation of an oral solution of a drug comprising the combination of the following four taste masking principles
   (1) a pharmaceutically acceptable acid,
   (2) a pharmaceutically acceptable sweetener,
   (3) a pharmaceutically acceptable salt and
   (4) a pharmaceutically acceptable flavor.

A third aspect of the invention in its first and broadest embodiment is directed to
(i) a water-soluble pharmaceutical capsule containing a powder formulation of a drug comprising an API susceptible to hydrolytic decomposition,

for use in treatment of patients who cannot swallow tablets, comprising dissolving the water-soluble capsule in
(ii) 50 to 250 ml of a suitable pharmaceutically acceptable solvent as a reconstitution medium contained in a pharmaceutically acceptable container, such as a bottle, 5 to 300% oversized by volume, and,
(iii) administration of a defined dosage by withdrawing the required volume of the oral solution from the bottle using an oral syringe of suitable volume and graduation to be connected with the bottle via an adapter plug, and administration of the defined dosage from the syringe orally to the patient.

Implicit to the third aspect of the invention is a method of administering the water-soluble pharmaceutical capsule containing a powder formulation of a drug comprising an API susceptible to hydrolytic decomposition to a patient who cannot swallow tablets, comprising

dissolving the water-soluble capsule in 50 to 250 ml of a suitable pharmaceutically acceptable solvent as a reconstitution medium contained in a pharmaceutically acceptable container 5 to 300% oversized by volume,

obtaining a defined dosage by withdrawing the required volume of the oral solution from the bottle using an oral syringe of suitable volume and graduation to be connected with the bottle via an adapter plug, and

administering the defined dosage from the syringe orally to the patient.

A fourth aspect of the invention in its first and broadest embodiment is directed to a process for preparing
(ii) a suitable pharmaceutically acceptable solvent as a reconstitution medium for preparation of an oral solution of a drug ready for administration, comprising the steps of successively dissolving the following four taste masking principles
   (1) a pharmaceutically acceptable acid,
   (2) a pharmaceutically acceptable sweetener,
   (3) a pharmaceutically acceptable salt and
   (4) a pharmaceutically acceptable flavor
   in purified water, preferably with stirring and at a temperature of 20 to 60° C., and adjusting to final weight by addition of purified water to obtain a bulk solution,
   optionally filtering the bulk solution and
   optionally filling the bulk solution in a pharmaceutically acceptable container, such as a bottle, and close the container.

The expression “a pharmaceutically acceptable acid” includes besides typically used acids like hydrochloric acid, phosphoric acid, citric acid, tartaric acid, succinic acid, fumaric acid, maleic acid, malic acid and the like also acidic preservatives such as sorbic acid or benzoic acid and the like.

Preferably 50 to 250 ml of the bulk solution are filled in a pharmaceutically acceptable container which is 5 to 300% oversized by volume, e.g. 100 ml of the bulk reconstitution medium are filled in 200 ml or 125 ml bottles.

### DETAILED DESCRIPTION OF THE INVENTION

The first aspect of the invention in a second embodiment is directed to a pharmaceutical kit comprising
(i) at least one water-soluble pharmaceutical capsule with capsule shells made of HPMC, PVA (polyvinylalcohol), starch or Pullulan (α-1,4-; α-1,6-glucan) containing a powder formulation comprising an API susceptible to hydrolytic decomposition, preferably packed in a plastic bottle, a plastic blister or an alu blister,
(ii) 50 to 150 ml of a suitable pharmaceutically acceptable solvent as a reconstitution medium comprising
   (a) 0.1% -5% by weight of one or more pharmaceutically acceptable artificial sweeteners or 0.1% -70% by weight of one or more pharmaceutically acceptable natural sweeteners or 0.1% -65% by weight of one or more pharmaceutically acceptable natural sweeteners and 0.1% -5% by weight of one or more pharmaceutically acceptable artificial sweeteners,
   (b) 0.01-1% by weight of one or more pharmaceutically acceptable acids, preferably acidic preservatives,
   (c) 0.01-1% by weight of one or more pharmaceutically acceptable flavors,
   (d) 0.1-1% by weight of one or more pharmaceutically acceptable salts or salty taste modifiers,
   (e) optionally up to 10-20% by weight of one or more texture modifiers,
   (f) optionally one or more antioxidants, such as ascorbic acid, butylhydroxytoluol (BHT) or butylhydroxyanisol (BHA),
   (g) optionally one or more stabilizers, such as EDTA,
   (h) optionally one or more pH modifiers such as a pharmaceutically acceptable acid, base or buffer, for adjustment of a physiologically acceptable pH, and
   (j) purified water as base solvent q.s. ad 100.0%,
   contained in a pharmaceutically acceptable container, such as a bottle, 5 to 100% oversized by volume preferably made of brown glass, for preparation of an oral solution comprising the API ready for administration with a shelf-life of the oral solution of up to 6 months at ambient temperature, and
(iii) at least one oral syringe of 0.5 to 60 ml volume and suitable graduation which can be connected with the bottle via an adapter plug, for dosing and administration,

and, optionally,
(iv) handling instructions comprising preparation of the oral API solution, measurement, withdrawal and administration of a dose.

Preferably the water-soluble pharmaceutical capsule has an approximate length of 20 to 30 mm to avoid inadvertent swallowing of the capsule, the capsule shells are made of HPMC and 1, 2, 3, 4 or 5 pharmaceutical capsules are packed in a polypropylene bottle with desiccant in the cap.

The second aspect of the invention in a second embodiment is directed to
(ii) a suitable pharmaceutically acceptable solvent as a reconstitution medium for preparation of an oral solution ready for administration comprising an API susceptible to hydrolytic decomposition, with a shelf-life of the oral solution of up to 6 months at ambient temperature, comprising
   (a) 0.1% -5% by weight of one or more pharmaceutically acceptable artificial sweeteners or 0.1% -70% by weight of one or more pharmaceutically acceptable natural sweeteners or 0.1% -65% by weight of one or more pharmaceutically acceptable natural sweeteners and 0.1% -5% by weight of one or more pharmaceutically acceptable artificial sweeteners,
   (b) 0.01-1% by weight one or more pharmaceutically acceptable acids, preferably acidic preservatives,
   (c) 0.01-1% by weight one or more pharmaceutically acceptable flavors,
   (d) 0.1-1% by weight one or more pharmaceutically acceptable salts or salty taste modifiers,
   (e) optionally up to 10-20% by weight one or more texture modifiers,
   (f) optionally one or more antioxidants, such as ascorbic acid, butylhydroxytoluol (BHT) or butylhydroxyanisol (BHA),
   (g) optionally one or more stabilizers, such as EDTA,
   (h) optionally one or more pH modifiers such as a pharmaceutically acceptable acid, base or buffer, for adjustment of a physiologically acceptable pH, and
   (j) purified water as base solvent q.s. ad 100.0%.

The third aspect of the invention in a second embodiment is directed to
(i) at least one water-soluble pharmaceutical capsule with capsule shells made of HPMC, PVA (polyvinylalcohol), starch or Pullulan (α-1,4-; α-1,6-glucan) containing a powder formulation comprising an API susceptible to hydrolytic decomposition, preferably packed in a plastic bottle, a plastic blister or an alu blister, for use in treatment of a patient who cannot swallow tablets, comprising dissolving the water-soluble capsule in
(ii) 50 to 150 ml of a suitable pharmaceutically acceptable solvent as a reconstitution medium comprising the combination of the following four taste masking principles
   (1) a pharmaceutically acceptable acid,
   (2) a pharmaceutically acceptable sweetener,
   (3) a pharmaceutically acceptable salt and
   (4) a pharmaceutically acceptable flavor,
   contained in a pharmaceutically acceptable container, such as a bottle, 5 to 300% oversized by volume, for preparation of an oral solution comprising the API ready for administration,
   or, more specifically, 50 to 150 ml of a suitable pharmaceutically acceptable solvent as a reconstitution medium comprising
   (a) 0.1% -5% by weight of one or more pharmaceutically acceptable artificial sweeteners or 0.1% -70% by weight of one or more pharmaceutically acceptable natural sweeteners or 0.1% -65% by weight of one or more pharmaceutically acceptable natural sweeteners and 0.1% -5% by weight of one or more pharmaceutically acceptable artificial sweeteners,
   (b) 0.01-1% by weight of one or more pharmaceutically acceptable acids, preferably acidic preservatives,
   (c) 0.01-1% by weight of one or more pharmaceutically acceptable flavors,
   (d) 0.1-1% by weight of one or more pharmaceutically acceptable salts or salty taste modifiers,
   (e) optionally up to 10-20% by weight of one or more texture modifiers,
   (f) optionally one or more antioxidants, such as ascorbic acid, butylhydroxytoluol (BHT) or butylhydroxyanisol (BHA),
   (g) optionally one or more stabilizers, such as EDTA,
   (h) optionally one or more pH modifiers such as a pharmaceutically acceptable acid, base or buffer, for adjustment of a physiologically acceptable pH, and
   (j) purified water as base solvent q.s. ad 100.0%,
   contained in a pharmaceutically acceptable container, such as a bottle, 5 to 300% oversized by volume, for preparation of an oral solution comprising the API ready for administration,
   and
(iii) administration of a defined dosage by withdrawing the required volume, e.g. 0.5 to 60 ml, of the oral solution from the bottle using an oral syringe of suitable volume and graduation to be connected with the bottle via an adapter plug, and administration of the defined dosage from the syringe orally to the patient.

Preferably the pharmaceutically acceptable solvent as a reconstitution medium is an aqueous solvent.

The fourth aspect of the invention in a second embodiment is directed to a process for preparing
(ii) a suitable pharmaceutically acceptable solvent as a reconstitution medium for preparation of an oral solution of a drug ready for administration, comprising an API susceptible to hydrolytic decomposition, with a shelf-life of the oral solution of up to 6 months at ambient temperature, comprising the steps

successively dissolving
(a) 0.1% -5% by weight of one or more pharmaceutically acceptable artificial sweeteners or 0.1% -70% by weight of one or more pharmaceutically acceptable natural sweeteners or 0.1% -65% by weight of one or more pharmaceutically acceptable natural sweeteners and 0.1% -5% by weight of one or more pharmaceutically acceptable artificial sweeteners,
(b) 0.01-1% by weight one or more pharmaceutically acceptable acids, preferably acidic preservatives,
(c) 0.01-1% by weight of one or more pharmaceutically acceptable flavors,
(d) 0.1-1% by weight of one or more pharmaceutically acceptable salts or salty taste modifiers,
(e) optionally up to 10-20% by weight one or more texture modifiers,
(f) optionally one or more antioxidants, such as ascorbic acid, butylhydroxytoluol (BHT) or butylhydroxyanisol (BHA),
(g) optionally one or more stabilizers, such as EDTA,
(h) optionally one or more pH modifiers such as a pharmaceutically acceptable acid, base or buffer, for adjustment of a physiologically acceptable pH,

in purified water as base solvent, preferably with stirring at a temperature of 20 to 60° C., preferably 20 to 40° C., and adjusting to final weight by addition of purified water as base solvent q.s. ad 100.0% to obtain a bulk solution,

optionally filtering the bulk solution and

optionally filling the bulk solution in pharmaceutically acceptable containers, such as bottles, 5 to 100% oversized by volume, preferably 5 to 30% or specifically 25% oversized, and close the containers.

A container or bottle 100% oversized by volume means that 100 ml of bulk solution is filled in a container or bottle of 200 ml volume.

The oral solution ready for administration comprising an API susceptible to hydrolytic decomposition may have a shelf-life of the oral solution of up to 6 months, of up to 3 months, of up to 4 weeks or of up to one week at ambient temperature.

APIs suitable to be used in the context of the invention may be selected from oncological small-molecule (NCE) drugs mentioned hereinbefore, preferably from reversible or irreversible binding EGFR inhibitors such as gefitinib, erlotinib, pelitinib, neratinib, afatinib, HKI-357, CI-1033 (canertinib), WZ 3146, WZ 4002, WZ 8040, dacomitinib, CO-1686, AZD9291, HM781-36B, and HM61713, or pharmaceutically acceptable salts thereof.

A second preferred subgroup of APIs suitable to be used in the context of the invention is selected from gefitinib, erlotinib, neratinib, afatinib, CI-1033 (canertinib), dacomitinib, CO-1686, and AZD9291, and HM61713, or pharmaceutically acceptable salts thereof.

A third preferred subgroup of APIs suitable to be used in the context of the invention is selected from neratinib, afatinib, dacomitinib, CO-1686 and AZD9291, whereas afatinib is particularly preferred, or pharmaceutically acceptable salts thereof. Most preferred is the dimaleate salt of afatinib (BIBW 2992 MA2).

Suitable sweeteners as components of the reconstitution medium may be selected from natural sweeteners such as sucrose, glucose, fructose, xylitol, maltitol, mannitol, and sorbitol, or from artificial sweeteners such as sucralose, aspartame, acesulfam-K, saccharin, saccharin-Na, Na-cyclamat, stevia extract and the like. A preferred sweetener is sucralose.

Suitable preservatives as components of the reconstitution medium may be selected from sorbic acid, K-sorbate, Na-benzoate, benzoic acid, parabens, methyl parabens, benzalkoniumchloride and the like. A preferred preservative is sorbic acid.

Suitable flavors as components of the reconstitution medium may be selected from e.g. strawberry, raspberry, currant, cream, cacao, chocolate, vanilla, cherry, tutti frutti, mint and the like, which may be used also in combination of up to 3 different flavors within a reconstitution medium. Preferred flavors are strawberry, cream, cacao and vanilla, or the combination of cream and strawberry flavor, as well as the combination of cacao and vanilla flavor.

Suitable salty taste modifiers as components of the reconstitution medium may be selected from NaCL or NaH₂PO₄, and the like. A preferred salty taste modifier is NaCL.

Suitable texture modifiers as components of the reconstitution medium may be selected from e.g. glycerol, soluble PVP, or cellulose derivatives such as hydroxyethylcellulose, hydroxypropylcellulose, methylcellulose or hydroxypropylmethylcellulose, and the like.

Suitable antioxidants as components of the reconstitution medium may be selected from ascorbic acid, butylhydroxytoluol (BHT) and butylhydroxyanisol (BHA) and the like. A preferred stabilizer is EDTA. As pH modifiers may be used suitable amounts of NaOH, HCL or NaH₂PO₄.

Suitable capsules that dissolve at room temperature within 30 min under occasionally shaking in the reconstitution medium according to the invention are, for instance, transparent HPMC hard shell capsules size 00, e.g. Vcaps Plus® available from Capsugel. Gelatine shells cannot be dissolved at room temperature since these capsules are only gelling. The composition of the reconstitution medium enables taste masking of bitter API. 100 ml of the reconstitution medium may be contained in a 125 ml bottle, with some free head space necessary to apply shear forces to the capsule during the dissolution process. Preferably the bottle is made from brown glass.

The handling instruction mentioned hereinbefore may comprise for example:

For preparation of the oral solution ready for administration:

Open the bottle containing the solvent for oral solution;

Open the plastic bottle containing capsules with the active ingredient for oral solution;

Transfer a defined number of capsules carefully into the solvent;

Mount the plastic adapter on the glass bottle and close the bottle with the screw cap;

Wait 15 min and shake the bottle vigorously for at least 10 seconds;

Wait 10 min and shake the bottle again vigorously for at least 10 seconds;

After waiting 5 min, gently shake the solution. The solution is ready to use. The solution might contain undissolved particles resulting from excipients of the capsule formulation which do not affect the quality of the drug product.

The handling instruction for measurement and withdrawal of a dose may comprise:

Gently shake the bottle;

Open the bottle;

Insert the oral syringe in the adapter (optionally referring to a picture);

Rotate the bottle including adapter and syringe upside down (optionally referring to a picture);

Withdraw the required volume. If air bubbles are visible, empty the syringe into the bottle and repeat the withdrawal of the required volume.

Administer the bubble-free solution orally to the patient.

Any of the formulation options defined hereinbefore exhibit unexpected good matching results >95% in placebo taste match model of e-tongue measurements, typical average match values have been seen in a range of up to 80%, particularly those comprising the combination of the four taste masking principles acid plus salt plus sweetener plus flavor revealed superior results, and specifically those described in the Examples.

In any aspects of the invention the patient may be a pediatric patient suffering from cancer, more specifically from

recurrent or refractory rhabdomyosarcoma with ErbB receptor family deregulation and/or the specific tumour type independent from ErbB deregulation testing status, or from

recurrent or refractory neuroectodermal tumours, i.e. high grade glioma (HGG), diffuse intrinsic pontine glioma (DIPG), low grade astrocytoma, neuroblastoma, ependymoma, medulloblastoma/primitive neuroectodermal tumour with ErbB receptor family deregulation and/or the specific tumour type independent from ErbB deregulation testing status,

rarely occurring in adult patients,

to be treated with a drug comprising afatinib or a pharmaceutically acceptable salt thereof, such as afatinib dimaleate, as the API, which is susceptible for hydrolytic decomposition.

The pediatric patient is a patient with an age of 6 months to 17 years, with defined subgroups of 6 months to 1 year, 6 months to 2 years, 6 months to 1 year, 1 to 3 years, 2 to 4 years, 4 to 8 years, and 8 to 17 years.

Afatinib film-coated tablets as described in WO 2009/147238 and afatinib capsules and solvent for oral solution according to the subject invention are considered age appropriate formulations covering the needs for treatment of pediatric patients of 6 months to 17 years with adequate dosing flexibility and patient convenience. The oral route of administration and once daily posology allow administration by caregivers outside the hospital setting to minimize the impact on daily activities, incl. participation in public life, e.g. school, of the paediatric patients. The intended dosing schedule ranging from 4 mg, applied as oral solution, to 60 mg, either applied as film-coated tablets or oral solution, is therefore well covered by the two formulations.

Volumes between 1.0 mL and 15.0 mL (equivalent to 4 mg to 60 mg dosage of afatinib), specifically of 1.0, 2.0, 3.0, 4.0, 5.0, 6.0, 7.0, 8.0, 9.0, 10.0, 11.0, 12.0, 13.0, 14.0, and 15.0 mL are expected to be applied, preferably once daily. Any of these dosages may be used as the total daily dosage for a patient, depending on the age and the specific needs of the patient. The daily total dosage of the drug may be separated into multiple single dosages administered over the day, preferably 2 or 3 single dosages. Dosing and administration is performed with an oral syringe suitable for the intended volume. The syringe is to be connected to the bottle via an adapter plug.

As decreased bioavailability of afatinib has been observed in adults ad fed state, the oral solution is not intended to be administered with food or drinks.

A relative bioavailability trial was performed in healthy adult volunteers comparing afatinib dimaleate administered as a drinking solution and as a 20 mg film-coated tablet (afatinib dimaleate, dose provided as the afatinib content). In this trial no significant differences were observed in the relative bioavailability of afatinib administered as a drinking solution and as a 20 mg film-coated tablet. In the drinking solution used in the trial the active substance was completely dissolved. The excipients in the drinking solution are not considered to have any impact on the pharmacokinetic behavior of afatinib.

The conclusions drawn for the drinking solution used for the study are considered to be representative for the pediatric formulation.

### EXAMPLES

### Example 1

Composition of afatinib 200 mg capsules (transparent HPMC hard shell capsule size 00 (Vcaps Plus® of Capsugel) containing dry-granulated BIBW 2992 MA2 as a white to slightly yellowish powder formulation):

| Ingredient | Amount [mg/capsule] | Function |
|---|---|---|
| Capsule fill | | |
| Afatinib dimaleate | 295.6 | Active ingredient |
| (Afatinib free base) | (200) | |
| Lactose, monohydrate | 274.4 | Filler |
| Crospovidone | 18.0 | Disintegrant |
| Magnesium stearate | 12.0 | Lubricant |
| Subtotal | 600.0 | |
| Capsule shell | | |
| HPMC capsule size 00, | 118 | Capsule shell |
| transparent | | |
| Subtotal | 118 | |
| Total | 718.0 | |

The dry-granulated BIBW 2992 MA2 powder formulation can be prepared in analogy as disclosed in WO 2009/147238.

Preferably two afatinib 200 mg capsules are packed in a 60 ml child-resistant polypropylene bottle with desiccant in the cap.

### Example 2

General solvent composition (reconstitution medium), Strawberry-cream reco-solvent option (125 ml brown glass bottle with child resistant cap filled with 100 ml of a clear solution).

| Composition | Quantity range |
|---|---|
| Sweetener (artificial) | Depending on type |
| e.g. Sucralose, Aspartame, Acesulfam-K, | 0.1%-5% |
| Saccharin-Na, Na-cyclamat etc. | standalone or in |
| | combination with natural |
| | sweetener |
| Natural sweetener (non cariogenic) | up to 60-70% standalone or |
| e.g. Maltitol, Sorbitol, Xylitol, Mannitol, | in combination with |
| Preservative | artificial sweetener 0.01-1% |
| e.g. Sorbic acid, Na-Benzoate, Benzoic acid, | acc. to specific properties |
| Parabens | |
| Antioxidants | Typically 0.01-0.5% |
| e.g. BHT, BHA, | |
| Ascorbic acid | Typically 0.01-0.1% |
| Stabilizer | Typically up to 0.15% |
| e.g. EDTA | |
| Flavor | Typically 0.01-1% |
| e.g. strawberry, raspberry, currant, cream, | |
| cacao, chocolate, vanilla, cherry, tutti frutti, | |
| mint, also in combination of up to 3 different | |
| flavors | |
| Salty taste modifier | 0.1-1% |
| (e.g. NaCL, NaH₂PO₄ etc.) | preferred 0.45-0.9% |
| Texture modifier | Depending on type up to |
| (e.g., Glycerol, Cellulose derivatives etc.) | 10-20% |
| pH-modifier | Amount sufficient to keep |
| e.g. HCL, NaOH, suitable buffer systems | formulation target pH after |
| | reconstitution at a range |
| | between pH 2.5-4.0 |
| Purified water as base solvent | q.s. ad final fill weight |

### Example 3

Solvent composition (reconstitution medium), Strawberry-cream reco-solvent option (125 ml brown glass bottle with child resistant cap filled with 100 ml of a clear solution).

| | | [mg/ | | Reference |
|---|---|---|---|---|
| Ingredients | [g/bottle] | bottle] | Function | to standards |
| Sucralose | 0.50 | 5.00 | Sweetener | USP-NF |
| Sorbic acid | 0.05 | 0.50 | Preservative | Ph. Eur./ |
| | | | | USP-NF |
| Sodium chloride | 0.90 | 9.00 | Taste modifier | Ph. Eur./USP |
| Cream flavor | 0.10 | 1.00 | Flavor | Company |
| | | | | standard |
| Strawberry flavor | 0.10 | 1.00 | Flavor | Company |
| | | | | standard |
| Purified water | 99.05 | 990.50 | Solvent | Ph. Eur./USP |
| Total weight | 100.70 | | | |

### Example 4

Solvent composition (reconstitution medium), strawberry-cream reco-solvent option (125 ml brown glass bottle with child resistant cap filled with 100 ml of a clear solution).

| | Low dose preservative | High dose preservative |
|---|---|---|
| | (0.05%) | (0.15%) |
| Composition | g/bottle | g/bottle |
| Sucralose | 0.5 | 0.5 |
| Sorbic acid | 0.05 | 0.15 |
| Sodium chloride | 0.9 | 0.9 |
| Cream flavor | 0.1 | 0.1 |
| Strawberry flavor | 0.1 | 0.1 |
| Purified water | q.s. ad 100.0 ml | q.s. ad 100.0 ml |

Strawberry/cream flavor variant showed excellent taste masking efficiency.

### Example 5

Solvent composition (reconstitution medium), cacao-vanilla reco-solvent option (125 ml brown glass bottle with child resistant cap filled with 100 ml of a clear solution).

| | Low dose preservative | High dose preservative |
|---|---|---|
| | (0.05%) | (0.15%) |
| Composition | g/bottle | g/bottle |
| Sucralose | 0.5 | 0.5 |
| Sorbic acid | 0.05 | 0.15 |
| Sodium chloride | 0.45 | 0.45 |
| Cacao flavor | 0.05 | 0.05 |
| Vanilla flavor | 0.2 | 0.2 |
| Purified water | q.s. ad 100.0 ml | q.s. ad 100.0 ml |

### Example 6

### Preparation of Afatinib Dimaleate Oral Solution Ready for Administration

The oral solution is prepared by dissolving two capsules in the supplied 100 ml solvent in the 125 ml brown glass bottle, resulting in an afatinib concentration of 4 mg/ml. The capsules must not be opened nor swallowed. The capsules are put into the bottle containing the solvent. The bottle is closed, and the capsules are dissolved by shaking the bottle manually in intervals.

The prepared solution is turbid and contains undissolved particles. The active substance is completely dissolved; the undissolved particles derive from the excipients (e.g. magnesium stearate, crospovidone) and do not impact the quality of the product or the dosing accuracy. The solution is stable for 4 weeks at 25° C. after preparation.

### Example 7

### Administration of Oral Solution

The prepared oral solution contains 4 mg/ml of afatinib. Based on body surface area dosage volumes between 1.0 and 15 ml might be applied. Dosing and administration is planned to be done using an oral syringe suitable for the intended volume. The syringe can be connected with the bottle via an adapter plug. The syringe should have a volume syringe of 0.5 to 60 ml volume and suitable graduation, e.g. in 0.1, 0.5 or 1.0 ml steps. Single use of an oral syringe (12 mL maximal volume) is suitable. For the possible use of dosing volumes greater than 12 mL the splitting of the dose re-using the pipette is considered acceptable. The syringe is foreseen to be cleaned with water after every use by filling and purging.

### Example 8

### Relative Bioavailability of Afatinib Final Formulation Tablet Compared to Oral Solution

A relative bioavailability (BA) trial was conducted to evaluate the relative BA of the 20 mg afatinib film-coated tablet to a 20 mg afatinib drinking solution. Geometric mean plasma concentrations of afatinib were slightly higher after administration of the drinking solution compared with the 20 mg film-coated tablet. However, the shape of the afatinib plasma concentration-time profiles was similar for the 20 mg film-coated tablet and the drinking solution tested. Maximum plasma concentrations were reached after 5 h (median tₘₐₓ) for both formulations. Also the mean residence time after peroral intake (MRT po) of afatinib was comparable for both formulations tested (20 mg film-coated tablet: 35.9 h versus drinking solution: 34.2 h), which might suggest that the mean absorption time of afatinib was equal for both formulations.

Intra-individual comparisons of Cₘₐₓ and AUC 0-∝ displayed a strong overlap of individual Cmax and AUC 0-∝ values between the 20 mg film-coated tablet and the drinking solution. The statistical evaluation of the relative bioavailability results are summarized in the table below. It should be noted that the trial was not powered to show bioequivalence between the formulations (N=22 healthy volunteers were treated in a cross-over design; considering the variability in PK observed in this trial, N=84 healthy volunteers would have been required to have a targeted power of 90% to show bioequivalence assuming a ratio of 0.95 for both formulations).

In this trial no significant differences were observed in the relative bioavailability of afatinib administered as a drinking solution and as a 20 mg film-coated tablet

Summary of Results from the Relative Bioavailability Trial 1200.35 20 mg Film-Coated Tablet Versus Drinking Solution

| | | | Geometric | Lower | Upper |
|---|---|---|---|---|---|
| | | | mean ratio | 90% CI | 90% CI |
| Test | Reference | Parameter | (%) | (%) | (%) |
| 20 mg film- | Drinking | Cₘₐₓ | 85.31 | 68.75 | 105.88 |
| coated | solution 20 mg | [ng/ml] | | | |
| tablet (FF | per bottle | AUC_{0-∞} | 92.24 | 76.30 | 111.51 |
| tablet) | | [ng · h/ml] | | | |

The 20 mg film-coated tablets (FF tablet) used in the trial is identical to the commercial 20 mg film-coated tablets except for the color of the film-coat and the embossment. Both formulations have been demonstrated to have the same dissolution profiles and are therefore expected to have the same pharmacokinetic behavior.

The drinking solution containing 20 mg of afatinib per bottle used in the trial was prepared by dissolving the drug substance in 80 ml of a solvent consisting of hydroxyethyl cellulose, poloxamer 188 and purified water. The active substance is completely dissolved. The excipients contained in the drinking solution did not show an impact on the pharmacokinetic behavior of afatinib.

1. A pharmaceutical kit comprising
(i) at least one water-soluble pharmaceutical capsule containing a powder formulation of a drug comprising an active pharmaceutical ingredient (API) susceptible to hydrolytic decomposition,
(ii) 50 to 250 ml of a suitable pharmaceutically acceptable solvent as a reconstitution medium contained in a pharmaceutically acceptable container 5 to 300% oversized by volume, for preparation of an oral solution comprising the API ready for administration with a shelf-life of the oral solution of up to 6 months at ambient temperature, and, optionally
(iii) an oral syringe of suitable volume and graduation which can be connected with the bottle via an adapter plug, for dosing and administration,
and, optionally,
(iv) handling instructions comprising preparation of the oral API solution, measurement, withdrawal and administration of a dose.

2. The pharmaceutical kit of claim 1 comprising
(i) at least one water-soluble pharmaceutical capsule with capsule shells made of HPMC, PVA (polyvinylalcohol), starch or Pullulan (α-1,4-; α-1,6-glucan) containing a powder formulation comprising an API susceptible to hydrolytic decomposition,
(ii) 50 to 150 ml of a suitable pharmaceutically acceptable solvent as a reconstitution medium comprising
(a) 0.1% -5% by weight of one or more pharmaceutically acceptable artificial sweeteners or 0.1% -70% by weight of one or more pharmaceutically acceptable natural sweeteners or 0.1% -65% by weight of one or more pharmaceutically acceptable natural sweeteners and 0.1% -5% by weight of one or more pharmaceutically acceptable artificial sweeteners,
(b) 0.01-1% by weight of one or more pharmaceutically acceptable acids,
(c) 0.01-1% by weight of one or more pharmaceutically acceptable flavors,
(d) 0.1-1% by weight of one or more pharmaceutically acceptable salts or salty taste modifiers,
(e) optionally up to 10-20% by weight of one or more texture modifiers,
(f) optionally one or more antioxidants,
(g) optionally one or more stabilizers,
(h) optionally one or more pH modifiers for adjustment of a physiologically acceptable pH, and
(j) purified water as base solvent q.s. ad 100.0%,
contained in a pharmaceutically acceptable container 5 to 100% oversized by volume for preparation of an oral solution comprising the API ready for administration with a shelf-life of the oral solution of up to 6 months at ambient temperature, and
(iii) at least one oral syringe of 0.5 to 60 ml volume and suitable graduation which can be connected with the bottle via an adapter plug, for dosing and administration,
and, optionally,
(iv) handling instructions comprising preparation of the oral API solution, measurement, withdrawal and administration of a dose.

3. The pharmaceutical kit of claim 1, wherein the API is selected from the group consisting of gefitinib, erlotinib, pelitinib, neratinib, afatinib, HKI-357, CI-1033 (canertinib), WZ 3146, WZ 4002, WZ 8040, dacomitinib, CO-1686, AZD9291, HM781-36B, and HM61713, or pharmaceutically acceptable salts thereof.

4. The pharmaceutical kit of claim 2, wherein the natural sweeteners are selected from the group consisting of sucralose, glucose, fructose, xylitol, maltitol, mannitol, and sorbitol, and the artificial sweeteners are selected from the group consisting of aspartame, acesulfam-K, saccharin, saccharin-Na, Na-cyclamat, and stevia extract,
the pharmaceutically acceptable acids are selected from the group consisting of hydrochloric acid, phosphoric acid, citric acid, tartaric acid, succinic acid, fumaric acid, maleic acid, malic acid, sorbic acid and benzoic acid,
the pharmaceutically acceptable flavors are selected from the group consisting of strawberry, raspberry, currant, cream, cacao, chocolate, vanilla, cherry, tutti frutti, and mint,
the pharmaceutically acceptable salts or salty taste modifiers are selected from the group consisting of NaCl and NaH₂PO₄,
the texture modifiers are selected from the group consisting of glycerol, soluble PVP (polyvinylpyrrolidone), and cellulose derivatives,
the antioxidants are selected from the group consisting of ascorbic acid, butylhydroxytoluol (BHT) and butylhydroxyanisol (BHA), and
the pH modifiers are selected from the group consisting of NaOH, HCL and NaH₂PO₄.

5. A pharmaceutically acceptable solvent comprising the combination of the following four taste masking principles
(1) a pharmaceutically acceptable acid,
(2) a pharmaceutically acceptable sweetener,
(3) a pharmaceutically acceptable salt and
(4) a pharmaceutically acceptable flavor.

6. The pharmaceutically acceptable solvent according to claim 5, comprising
(a) 0.1% -5% by weight of one or more pharmaceutically acceptable artificial sweeteners or 0.1% -70% by weight of one or more pharmaceutically acceptable natural sweeteners or 0.1% -65% by weight of one or more pharmaceutically acceptable natural sweeteners and 0.1% -5% by weight of one or more pharmaceutically acceptable artificial sweeteners,
(b) 0.01-1% by weight one or more pharmaceutically acceptable acids,
(c) 0.01-1% by weight one or more pharmaceutically acceptable flavors,
(d) 0.1-1% by weight one or more pharmaceutically acceptable salts or salty taste modifiers,
(e) optionally up to 10-20% by weight one or more texture modifiers,
(f) optionally one or more antioxidants,
(g) optionally one or more stabilizers,
(h) optionally one or more pH modifiers for adjustment of a physiologically acceptable pH, and
(j) purified water as base solvent q.s. ad 100.0%.

7. The pharmaceutically acceptable solvent according to claim 6, wherein the natural sweeteners are selected from the group consisting of sucralose, glucose, fructose, xylitol, maltitol, mannitol, and sorbitol, and the artificial sweeteners are selected from the group consisting of aspartame, acesulfam-K, saccharin, saccharin-Na, Na-cyclamat, and stevia extract,
the pharmaceutically acceptable acids are selected from hydrochloric acid, phosphoric acid, citric acid, tartaric acid, succinic acid, fumaric acid, maleic acid, malic acid, sorbic acid and benzoic acid,
the pharmaceutically acceptable flavors are selected from the group consisting of strawberry, raspberry, currant, cream, cacao, chocolate, vanilla, cherry, tutti frutti, and mint,
the pharmaceutically acceptable salts or salty taste modifiers are selected from the group consisting of NaCl and NaH₂PO₄,
the texture modifiers are selected from the group consisting of glycerol, soluble PVP (polyvinylpyrrolidone), and cellulose derivatives,
the antioxidants are selected from the group consisting of ascorbic acid, butylhydroxytoluol (BHT) and butylhydroxyanisol (BHA), and
the pH modifiers are selected from the group consisting of NaOH, HCL and NaH₂PO₄.

8. A method of administering a water-soluble pharmaceutical capsule containing a powder formulation of a drug comprising an API susceptible to hydrolytic decomposition to a patient who cannot swallow tablets, comprising
dissolving the water-soluble capsule in 50 to 250 ml of a suitable pharmaceutically acceptable solvent as a reconstitution medium contained in a pharmaceutically acceptable container 5 to 300% oversized by volume,
obtaining a defined dosage by withdrawing the required volume of the oral solution from the bottle using an oral syringe of suitable volume and graduation to be connected with the bottle via an adapter plug, and
administering the defined dosage from the syringe orally to the patient.

9. The method according to claim 8, wherein the capsule shells are made of HPMC, PVA (polyvinylalcohol), starch or Pullulan (α-1,4-; α-1,6-glucan), and wherein the reconstitution medium has a volume of 50 to 150 ml and comprises the combination of the following four taste masking principles
(1) a pharmaceutically acceptable acid,
(2) a pharmaceutically acceptable sweetener,
(3) a pharmaceutically acceptable salt and
(4) a pharmaceutically acceptable flavor.

10. The method according to claim 9, wherein the reconstitution medium comprises
(a) 0.1% -5% by weight of one or more pharmaceutically acceptable artificial sweeteners or 0.1% -70% by weight of one or more pharmaceutically acceptable natural sweeteners or 0.1% -65% by weight of one or more pharmaceutically acceptable natural sweeteners and 0.1% -5% by weight of one or more pharmaceutically acceptable artificial sweeteners,
(b) 0.01-1% by weight of one or more pharmaceutically acceptable acids,
(c) 0.01-1% by weight of one or more pharmaceutically acceptable flavors,
(d) 0.1-1% by weight of one or more pharmaceutically acceptable salts or salty taste modifiers,
(e) optionally up to 10-20% by weight of one or more texture modifiers,
(f) optionally one or more antioxidants,
(g) optionally one or more stabilizers,
(h) optionally one or more pH modifiers for adjustment of a physiologically acceptable pH, and
(j) purified water as base solvent q.s. ad 100.0%.

11. The method according to claim 8, wherein the API is selected from the group consisting of gefitinib, erlotinib, pelitinib, neratinib, afatinib, HKI-357, CI-1033 (canertinib), WZ 3146, WZ 4002, WZ 8040, dacomitinib, CO-1686, AZD9291, HM781-36B, and HM61713, or pharmaceutically acceptable salts thereof.

12. The method according to claim 10, wherein the natural sweeteners are selected from the group consisting of sucralose, glucose, fructose, xylitol, maltitol, mannitol, and sorbitol, and the artificial sweeteners are selected from the group consisting of aspartame, acesulfam-K, saccharin, saccharin-Na, Na-cyclamat, and stevia extract,
the pharmaceutically acceptable acids are selected from the group consisting of hydrochloric acid, phosphoric acid, citric acid, tartaric acid, succinic acid, fumaric acid, maleic acid, malic acid, sorbic acid and benzoic acid,
the pharmaceutically acceptable flavors are selected from the group consisting of strawberry, raspberry, currant, cream, cacao, chocolate, vanilla, cherry, tutti frutti, and mint,
the pharmaceutically acceptable salts or salty taste modifiers are selected from the group consisting of NaCl and NaH₂PO₄,
the texture modifiers are selected from the group consisting of glycerol, soluble PVP (polyvinylpyrrolidone), and cellulose derivatives,
the antioxidants are selected from the group consisting of ascorbic acid, butylhydroxytoluol (BHT) and butylhydroxyanisol (BHA), and
the pH modifiers are selected from the group consisting of NaOH, HCL and NaH₂PO₄.

13. The method according to claim 8, wherein the patient is a pediatric patient with an age of 6 months to 17 years, suffering from cancer, and the API is afatinib or a pharmaceutically acceptable salt thereof.

14. The method according to claim 13, wherein the cancer is selected from the group consisting of recurrent or refractory rhabdomyosarcoma with ErbB receptor family deregulation and/or the specific tumour type independent from ErbB deregulation testing status, recurrent or refractory neuroectodermal tumours, diffuse intrinsic pontine glioma (DIPG), low grade astrocytoma, neuroblastoma, ependymoma, medulloblastoma/primitive neuroectodermal tumour with ErbB receptor family deregulation and the specific tumour type independent from ErbB deregulation testing status.

15. A process for preparing a pharmaceutically acceptable solvent as a reconstitution medium for preparation of an oral solution of a drug ready for administration, comprising the steps of:
successively dissolving the following four taste masking principles
(1) a pharmaceutically acceptable acid,
(2) a pharmaceutically acceptable sweetener,
(3) a pharmaceutically acceptable salt and
(4) a pharmaceutically acceptable flavor
in purified water,
adjusting to final weight by addition of purified water to obtain a bulk solution,
optionally filtering the bulk solution, and
optionally filling the bulk solution in a pharmaceutically acceptable container.

16. The process of claim 15 wherein the reconstitution medium comprises
(a) 0.1% -5% by weight of one or more pharmaceutically acceptable artificial sweeteners or 0.1% -70% by weight of one or more pharmaceutically acceptable natural sweeteners; or 0.1% -65% by weight of one or more pharmaceutically acceptable natural sweeteners and 0.1% -5% by weight of one or more pharmaceutically acceptable artificial sweeteners,
(b) 0.01-1% by weight one or more pharmaceutically acceptable acids,
(c) 0.01-1% by weight of one or more pharmaceutically acceptable flavors,
(d) 0.1-1% by weight of one or more pharmaceutically acceptable salts or salty taste modifiers,
(e) optionally up to 10-20% by weight one or more texture modifiers,
(f) optionally one or more antioxidants,
(g) optionally one or more stabilizers,
(h) optionally one or more pH modifiers for adjustment of a physiologically acceptable pH, and
(i) purified water as base solvent,
the process further comprising successively dissolving the components (a)-(h) in the purified water (i),
adjusting to final weight by addition of the purified water as base solvent q.s. ad 100.0% to obtain a bulk solution,
optionally filtering the bulk solution and
optionally filling the bulk solution in pharmaceutically acceptable containers 5 to 100% oversized by volume.

17. The process of claim 16, wherein the natural sweeteners are selected from the group consisting of sucralose, glucose, fructose, xylitol, maltitol, mannitol, and sorbitol, and the artificial sweeteners are selected from the group consisting of aspartame, acesulfam-K, saccharin, saccharin-Na, Na-cyclamat, and stevia extract,
the pharmaceutically acceptable acids are selected from the group consisting of hydrochloric acid, phosphoric acid, citric acid, tartaric acid, succinic acid, fumaric acid, maleic acid, malic acid, sorbic acid and benzoic acid,
the pharmaceutically acceptable flavors are selected from the group consisting of strawberry, raspberry, currant, cream, cacao, chocolate, vanilla, cherry, tutti frutti, and mint,
the pharmaceutically acceptable salts or salty taste modifiers are selected from the group consisting of NaCl and NaH₂PO₄,
the texture modifiers are selected from the group consisting of glycerol, soluble PVP (polyvinylpyrrolidone), and cellulose derivatives,
the antioxidants are selected from the group consisting of ascorbic acid, butylhydroxytoluol (BHT) and butylhydroxyanisol (BHA), and
the pH modifiers are selected from the group consisting of NaOH, HCL and NaH₂PO₄.
